(19) United States
(12) Patent Application Publication
(10) Pub. No.: US 2007/0276285 A1
(43) Pub. Date: Nov. 29, 2007

(54) System and Method for Customized Training to Understand Human Speech Correctly with a Hearing Aid Device

(76) Inventors: Mark Burrows, Princeton, NJ (US); John Cronin, Jericho, VT (US); Nancy Edwards, Jericho, VT (US); Justin Kunz, South Burlington, VT (US); John Anthony Singarayar, Skillman, NJ (US)

Correspondence Address: Norris McLaughlin & Marcus, P O Box 1018, Somerville, NJ, 08876, US

(21) Appl. No.: 10/562,091

(22) PCT Filed: Jun. 24, 2004
(86) PCT No.: PCT/US04/20387

§ 371 Date: Aug. 7, 2007

Related U.S. Application Data
(60) Provisional application No. 60/482,159, filed on Jun. 24, 2003.

(51) Int. Cl. A61B 5/00

(52) U.S. Cl. 600/559

(57) ABSTRACT

A data storage media including customized hearing training information is created using troublesome content identified based on testing of an individual's hearing profile. The identified content, and amplification factors associated with the hearing profile and corresponding to the identified content, are stored on the media. The individual can readily access the media, which can be portable, to generate sound output representative of the identified content without, and adjusted by, the corresponding amplification factor, such that the individual can train his brain on the identified troublesome content before receiving a hearing aid.

### CROSS REFERENCE TO RELATED APPLICATION

This application claims the benefit of U.S. Provisional Application No. 60/482,159 filed Jun. 24, 2003, assigned to the assignee of this application and incorporated by reference herein.

### FIELD OF THE INVENTION

The present invention relates to training a individual to understand human speech correctly with a hearing aid device, and more particularly, to inexpensively and quickly creating a customized hearing training product that an individual can use to train on a series of words and sentences affected by the individual's hearing profile in preparation for the experience of hearing and listening when using a hearing aid.

### BACKGROUND OF THE INVENTION

According to the National Institute on Deafness and Other Communication Disorders (NIDCD), approximately 28 million Americans have hearing loss and approximately 1.4 million individuals over the age of three are deaf in both ears. It has been estimated that hearing loss could cost society as much as $297,000 over the lifetime of an affected individual. As the baby boomer population ages, the impact of hearing loss becomes even more serious and widespread because there is a natural onset of hearing loss after the age of 35.

Unfortunately, the majority of the population is either unaware of or does not seek assistance for their hearing difficulties. It has been reported that three out of five older Americans and six out of seven middle-aged Americans do not use a hearing-aid device. The following are reasons for lack of use of a hearing aid by those who would benefit from their use. First, the individual may not understand the severity of his hearing loss. As the brain continuously adjusts over time to compensate for such loss, it trains itself to believe it hears everything correctly. In addition, the price of a hearing aid makes it unavailable for many. It has been found that, in the U.S. alone, 7 million individuals who would benefit from a hearing aid cannot afford one. Finally, many are concerned with the negative images associated with wearing such a device.

Until the mid-1980s, traditional hearing aids were based on analog technology and merely acted as amplifiers. In the mid-1990s, ten years after their initial introduction, digital-based aids became the accepted standard. A digital signal processor (DSP) was added directly to the hearing aid device and could be placed either inside or behind the ear. This change in technology meant that an audiologist could perform a hearing test on an individual and customize the aid by programming the DSP. This improved the individual's hearing because the DSP could selectively amplify identified troublesome frequency ranges.

U.S. Pat. No. 6,289,310, incorporated by reference herein, describes a prior art technique for screening an individual's ability to process acoustic events. The '310 patent provides sequences (or trials) of acoustically processed target and distracter phonemes to a subject for identification. The acoustic processing includes amplitude emphasis of selected frequency envelopes, stretching (in the time domain) of selected portions of phonemes and phase adjustment of selected portions of phonemes relative to a base frequency. After a number of trials, a profile for an individual is developed that indicates whether the individual's ability to process acoustic events is within a normal range, and if not, what processing can provide the individual with optimal hearing. The individual's profile can then be used by a listening or processing device to particularly emphasize, stretch or otherwise manipulate an audio stream to provide the individual with an optimal chance of distinguishing between similar acoustic events.

The standard hearing test usually conducted upon an individual is based solely on frequency versus amplitude. Upon receiving and using the hearing aid, the individual may have difficulty with specific words and think that the hearing aid is faulty. In reality, there are specific words that are difficult for the individual to hear correctly until the individual relearns them while using the hearing aid. Oftentimes, these difficulties experienced by the individual can generate a level of frustration that causes the individual to abandon use of the hearing aid altogether.

Prior art techniques for determining troublesome content for an individual based on the individual's hearing profile typically are not relatively low cost, and also cannot be used to customize a training program for an individual quickly and at a relatively low cost. In addition, the prior art does not provide an individual with a means to illustrate to family and friends what hearing is like both before and after he receives the hearing aid.

It has been found that a substantial percentage of individuals who order a hearing aid, and perform some form of hearing training before receiving the hearing aid, decide to cancel the hearing aid order even before the hearing aid is received. The cancellations of these hearing aid orders have been attributed to the lack of a satisfactory hearing aid training means in the prior art. It is estimated that 20% of hearing aid orders are cancelled before an individual ever receives a hearing aid, and an even larger number of hearing aids go unused because the consumer does not give his brain time to readjust using the hearing aid.

Therefore, there exists a need for a customized hearing training product that can be created quickly, efficiently and inexpensively, is easy and inexpensive to access and addresses troublesome words and sentences within certain frequency and amplitude ranges, which are identified based on an individual's hearing profile, to prepare the individual for what hearing and listening will be like when using a hearing aid

### SUMMARY OF THE INVENTION

In accordance with the present invention, a customized hearing training product for training an individual on specific, troublesome content, which is identified based on the individual's hearing profile, is created quickly and inexpensively on an inexpensive and readily accessible data storage media, such as a portable CD or an Internet website. A customized hearing training computer system initially maps an individual's hearing profile based on hearing testing results. From the hearing profile, the system identifies troublesome content and corresponding amplification factors. The system then stores the identified content with the corresponding amplification factors onto a data storage media. The media with the identified content and amplification factors can be created relatively quickly following the testing at relatively low cost, such that the individual can quickly access the media using a conventional data storage media playback device to begin training his brain before he uses a hearing aid, thus minimizing the returns of hearing aids.

In a preferred embodiment, a customized hearing training computer system collects frequency and amplitude hearing loss data for an individual by performing a frequency versus amplitude hearing test on the individual. Based on the frequency and amplitude data, the system generates a hearing loss profile map including frequencies requiring amplification and associated amplification factors. The system then generates, based on the hearing profile map, training word units using a troublesome word database contained in or coupled to the system. The troublesome word database includes a plurality of words, where each of the words includes at least one frequency component and is indexed in accordance with the at least one frequency component. The training units include a troublesome word from the word database having at least one frequency component substantially equal to one, or within the range, of the frequencies requiring amplification in the individual's hearing profile map. In addition, for each training unit containing a specific troublesome word, there is a corresponding training unit including the specific word and the amplification factor for the one, or the range, of the frequencies for the word requiring amplification. The system then stores the training units on a data storage medium. The training units are stored to provide that a conventional sound output generating playback device generates sound output first as a normal version and then as a modified version of a specific word. The normal version sound output is without any amplification, and the modified version sound output includes selected amplification of the word based on the amplification factor.

In a further preferred embodiment, the media contains a training unit including a troublesome word as part of a sentence, and another training unit including the troublesome word as part of the sentence and an amplification factor for the troublesome word.

In still a further preferred embodiment, the training units are stored on the media such that the individual can selectively generate, for at least one of the training units, the normal or modified version sound output of the word.

In another preferred embodiment, the media includes a test word list for each of the troublesome words contained in a training unit stored on the media. A user accesses the training information stored on the media using a playback device, such a conventional computer including sound output means, such as a speaker, a display monitor, a mouse and keyboard, that executes a software application, which also is included in the media. The playback device generates, from at least a first training unit, sound output as a troublesome word modified by the corresponding amplification factor. The playback device then retrieves from the media a test word list corresponding to the troublesome word of the at least first training unit and displays the list on a monitor. The test list includes the troublesome word and words that sound similar to the troublesome word. The playback device again generates the sound output of the troublesome word modified by the corresponding amplification factor if the playback device does not receive an input from the user that correctly identifies the troublesome word from the list of test words.

### BRIEF DESCRIPTION OF THE DRAWINGS

Other objects and advantages of the present invention will be apparent from the following detailed description of the presently preferred embodiments, which description should be considered in conjunction with the accompanying drawings in which like references indicate similar elements and in which:

FIG. 1 is a high level system diagram of a low-cost hearing testing system that collects user information in accordance with the present invention.

FIG. 2 is a table showing an individual's hearing profile at specific amplitudes for numerous frequencies and the amplification factor needed to adjust hearing to a normal level.

FIG. 3 is a table showing words and sentences affected by an individual's hearing profile for specific frequencies at low-pass, band-pass, high-pass and notch hearing types.

FIG. 4 is a high level system diagram of a computer system that, in accordance with the present invention, creates an audio training CD and communicates with databases containing information to be stored on the CD.

FIG. 5 is a flow chart showing how an individual would interact with the audio training CD created by the system of FIG. 4.

### DETAILED DESCRIPTION OF THE INVENTION

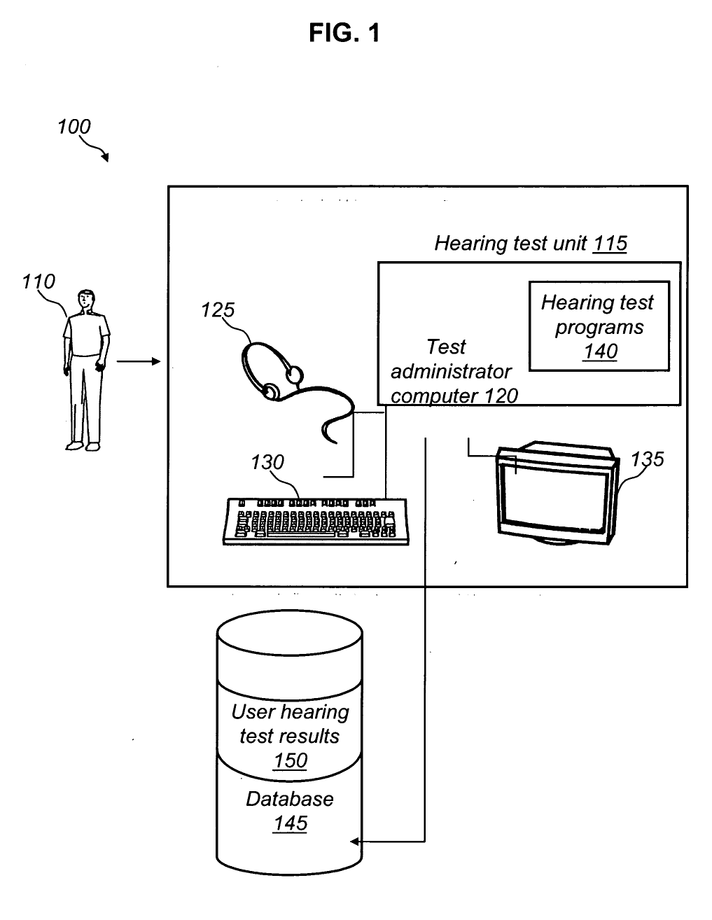

FIG. 1 is a high level diagram of a system 100 for collecting individual hearing profile information that can be used to generate hearing aid training data for storage on a data storage media in accordance with the present invention. Referring to FIG. 1, the system 100 includes a user 110, a hearing test unit 115, a test administrator computer 120, a pair of headphones 125, a keyboard 130, a monitor 135, a series of hearing test programs 140, a database 145 and a plurality of user hearing test results 150.

User 110 represents an individual on whom a hearing test is to be administered. Hearing test unit 115 includes a test administrator computer 120, which includes conventional headphones 125, conventional keyboard 130 and conventional monitor 135, all used for testing. For example, conventional monitor 135 can graphically display test frequencies and amplitudes for user 110, while user 110 is being tested.

Test administrator computer 120 executes a series of current hearing test programs 140 and may store the results of the tests. Test administrator computer 120 is also responsible for communicating with database 145. Database 145 is a central database repository for storing user hearing test results 150 about user 110 or any other test subject, which can later be reused. Database 145 can store an infinite number of individual hearing test results, and all of these results would be accessible using test administrator computer 120 or any other system linked to database 145.

In operation, user 110 wears headphones 125 and uses keyboard 130 and monitor 135 to take a hearing test using test administrator computer 120 and the series of hearing test programs 140. Individual results 150 of hearing test programs 140 are stored in database 145, which can be either located within test administrator computer 120 or centrally located.

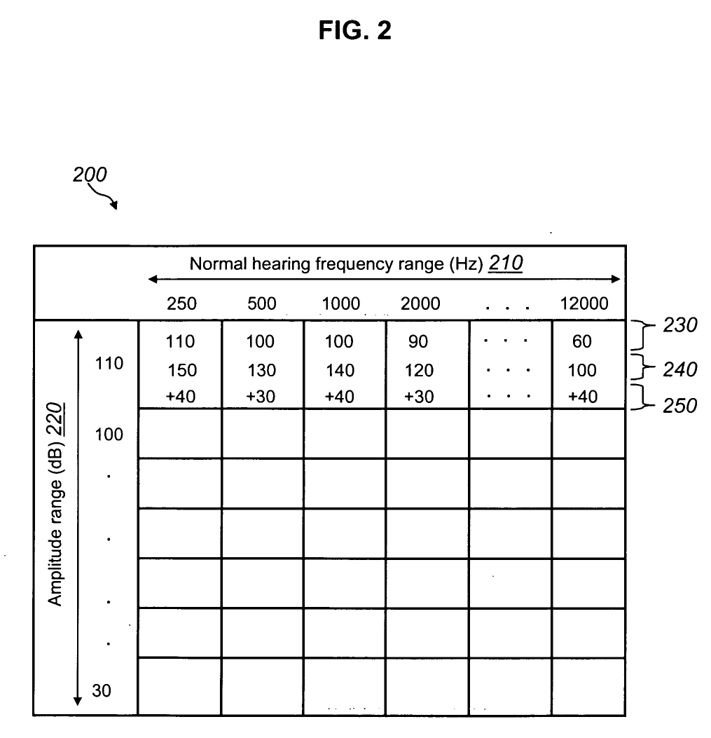

FIG. 2 illustrates a table 200 showing details of an individual's hearing profile and amplification factors that are needed to adjust hearing to a normal level. Referring to FIG. 2, the table 200 includes a normal hearing frequency range 210, an amplitude range 220, an example of values for individual hearing 230, an example of values for normal hearing 240 and an amplification factor 250. As is well known, humans hear at frequencies ranging from 15 to 20,000 hertz (Hz). Normal hearing frequency range 210 shows a smaller range from 250 to 12,000 Hz. During a hearing test performed, for example, using the test computer 20 of the system 100, an audiologist may choose to test sounds of different frequency ranges across a series of amplitudes. Amplitude range 220 shows a typical range of 30 to 110 decibels (dB). Individual hearing 230 shows an example of decibel levels by frequency that an individual may hear at 110 dB. Normal hearing 240 shows an example of the decibel levels by frequency that the individual should hear at 110 dB. Amplification factor 250 shows the difference between the values of individual hearing 230 and normal hearing 240 at 110 dB. An audiologist would adjust this individual's hearing aid by programming the DSP using amplification factor 250.

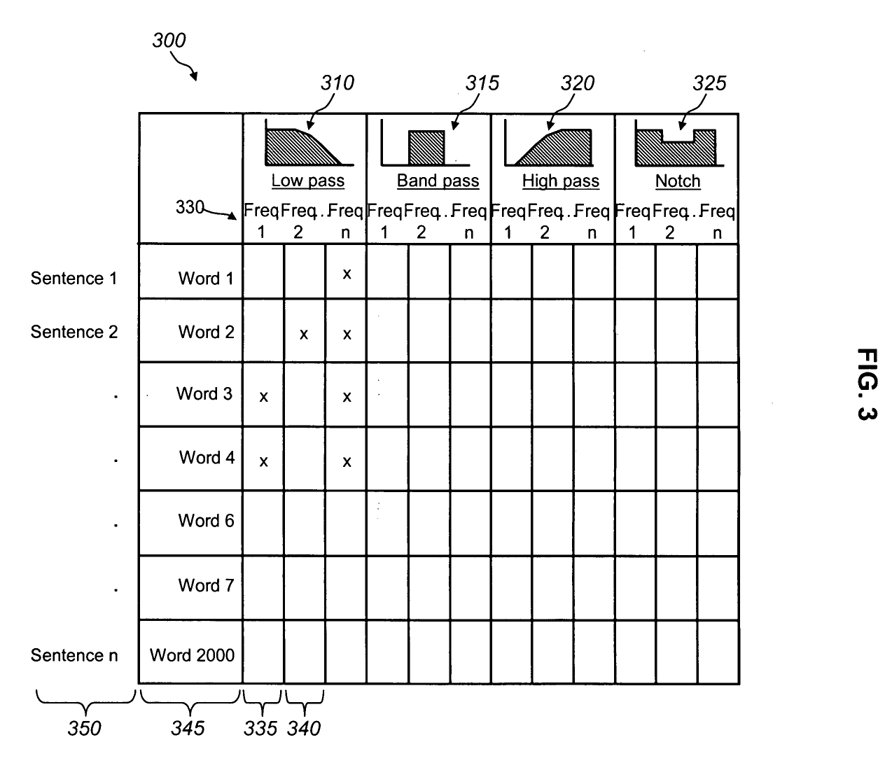

FIG. 3 illustrates an exemplary table 300 showing content identified as being affected by an individual's hearing profile. Referring to FIG. 3, the table 300 includes a low pass chart 310, a band pass chart 315, a high pass chart 320, a notch chart 325, a range of frequencies 330, a list of words checked for frequency-1 335, a list of words checked for frequency-2 340, a series of words 345 and a series of sentences 350.

Chart 310 in the table 300 is used in connection with a patient having a low pass spectrum of hearing. The patient's ear acts as a low pass filter, which means the patient has fairly good hearing between approximately 250 Hz and approximately 4000 Hz. Above these frequencies, the patient's perception of frequencies is filtered out or minimized.

Chart 315 in the table 300 is used in connection with a patient having a band pass spectrum of hearing. The patient's ear acts as a band pass filter, which means that the patient has fairly good hearing between approximately 4000 Hz and approximately 8000 Hz. Outside this range of frequencies, the patients' perception of frequencies is filtered out or minimized.

Chart 320 in the table 300 is used in connection with a patient having a high pass spectrum of hearing. The patient's ear acts as a high pass filter, which means that the patient has fairly good hearing between approximately 8000 Hz and approximately 12,000 Hz. Below these frequencies, the patients' perception of frequencies is filtered out or minimized.

Chart 325 in the table 300 is used in connection with a patient having a notch spectrum of hearing. The patient's ears act as a notch filter, which means the patient has fairly good hearing between approximately 250 Hz and approximately 4000 Hz and between approximately 8000 Hz and approximately 12,000 Hz, but not between approximately 4000 Hz and approximately 8000 Hz. In the “notch” of this range of frequencies, the patient's perception of frequencies is filtered out or minimized.

Referring to FIG. 2, the values of individual hearing 230 of table 200 from an individual's hearing loss profile map determine whether an individual would, for example, fall into one of four categories of hearing types, namely, low pass, band pass, high pass or notch as shown in FIG. 3. For purposes of illustration, in the exemplary table 300 it is assumed that the individual's ear behaves as a low pass filter. Based on range of frequencies 330, series of words 345 are marked as troublesome within that particular frequency for the individual. For example, Words 1, 2, 3, and 4 are troublesome words for a person with low pass hearing, whereas Words 6 and 7, etc., are not. Therefore, an individual may need further training on Words 1, 2, 3, and 4 before a hearing aid is used.

In table 300, each hearing type is further divided into a plurality of frequencies (1 through n) to provide that an individual's difficulties with certain words can be fine tuned. Continuing with the example, Word 1 is a troublesome word in frequency n and Word 2 is a troublesome word for frequency 2. The audiologist, thus, can uniquely identify words in a hearing type, such as low pass, high pass, etc., and even words within a hearing type, such as low pass, that could be troublesome for an individual to understand.

It is well known that words are patterns of frequency versus amplitude over time that have unique pattern signatures, called phonemes, that allow individuals to understand speech. In effect, the brain is trained over time and acts as a real-time DSP and lookup table system that match the pattern signature with a word. Many times, as an individual loses hearing in a certain range, certain words become troublesome to hear and the individual continually asks someone to repeat these words. In essence, the individual is retraining his or her brain. The word is often repeated in a sentence that provides more context for the brain to be retrained. Although the number of words that a human can understand can be quite large (hundreds of thousands), the number of words used in normal vocabulary (95% of normal usage) is about 2000 to 3000 words. These words readily can be included in the table 300. Thus, table 300 can easily be generated to encompass 95% of the words a human would hear. In addition, the words in the table 300 can easily be processed through a DSP to define most of the frequency range. The words can then be mapped into table 300 against frequency ranges that could be troublesome. This information is vital if training used with various types of hearing loss is required.

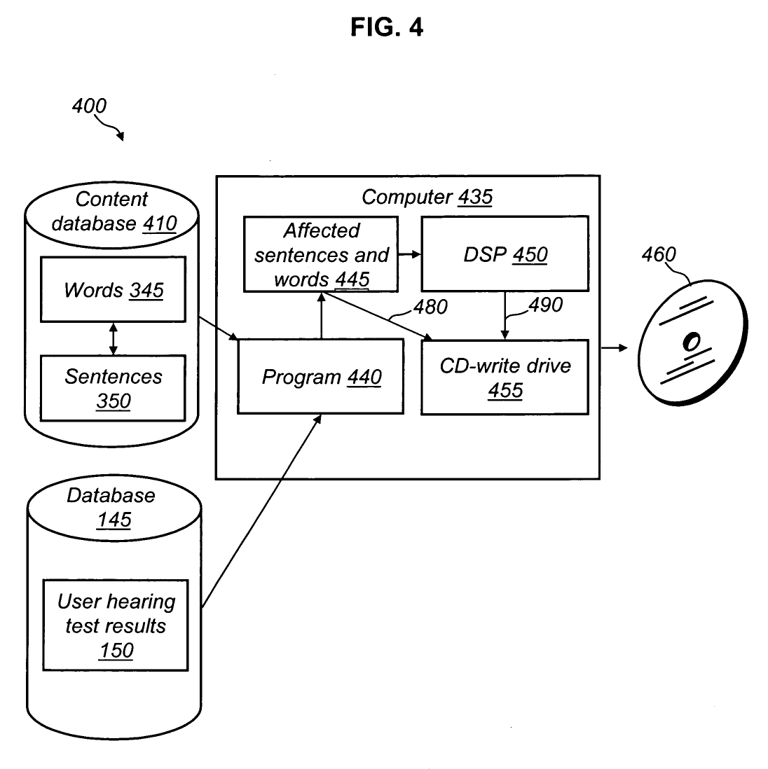

It is further understood that, for all words 345 in table 300, a sentence could be defined to add context to understanding the word. Just as the user might ask a speaker to repeat a sentence, the individual could play a pre-stored sentence over and over again. In the series of sentences 350, a single sentence may contain one or more words 345. Furthermore, a single word 345 may have multiple related sentences 350. The associations between a single word and multiple related sentences are described further in FIG. 4.

FIG. 4 shows a high level diagram of a system 400 for creating an audio training CD including training words and sentences that are customized to an individual's hearing profile determined from hearing aid testing. Referring to FIG. 4, the system 400 includes a content database 410, a group of words 345, a group of sentences 350, database 145, user hearing test results 150, a conventional computer 435, a program 440, an example of affected sentences and words 445, a DSP 450, a CD-write drive 455 and a CD 460.

Content database 410 contains a repository of all words 345 and sentences 350 that cause hearing difficulties. Database 145 contains user hearing test results 150, shown as individual hearing 230 values in FIG. 2 and measured using system 100 of FIG. 1. Computer 435 contains and executes program 440 that essentially performs the association between individual hearing 230 values as shown in FIG. 2 and words 345 and sentences 350 as shown in FIG. 3. Program 440 can write these associated words and sentences, shown as affected sentences and words 445 in FIG. 4, and record them normally, i.e., without a corresponding amplification factor 250 also identified in table 200 of FIG. 2, to CD-write drive 455 through a path 480. Program 440 can also take affected words and sentences 445 and process them through DSP 450 using the corresponding amplification factor 250 from the table 200 to record them to CD-write drive 455 through a path 490. The affected content, alone or accompanied by a corresponding amplification factor, are preferably arranged in training word units when recorded to CD-write drive 455. CD-write drive 455 stores the training words units representatively onto data storage units or tracks of CD 460. In a preferred embodiment, each affected word and each affected sentence is contained in a first training word unit without any corresponding amplification factor and also in a second training word unit with a corresponding amplification factor, and the system 400 stores the first and second training units for a same affected word or sentence, respectively, on consecutive tracks of the CD 460.

In an alternative preferred embodiment, CD 460 and CD-write drive 455 can be replaced by an alternative communication means such as the Internet. In this embodiment, program 440 can transfer affected sentences and words 445, with and without amplification factor 250, through DSP 450 to a user through the Internet. Use of the Internet provides for a higher level of interaction with the individual than CD 460, because information supplied by the individual can be immediately fed back and stored into database 145. In alternative preferred embodiments, the storage medium is any conventional fixed data storage media, such as a hard drive maintained at a central database remotely accessible over the Internet, or a portable data storage media, such as a memory stick, CD, CD-ROM, DVD or the like.

Thus, through use of the systems 400 and 100, an audiologist could perform a hearing exam with relative ease, inexpensively and quickly, and then immediately generate a hearing aid training product in the form of an audio training CD that is tailored specifically to the hearing profile of a consumer. The cost of testing the individual to create a customized training product would be relatively low, as the same equipment and databases can be used to test many individuals and generate data to be written onto a storage media. Further, the storage media on which hearing aid training data can be readily stored for easy access itself is relatively low cost, such as a blank CD or a website accessible by the individual over the Internet.

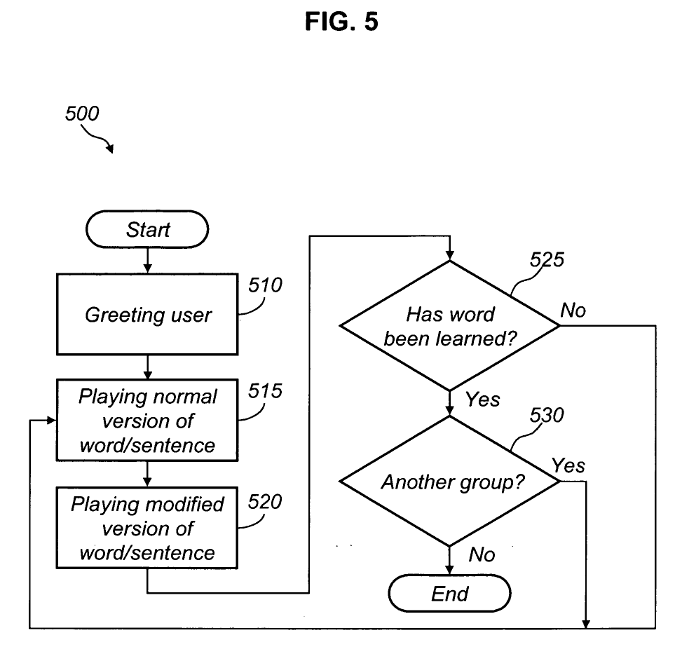

FIG. 5 illustrates a preferred method 500 of using CD 460, as generated by the system 400, for training an individual to understand human speech correctly in accordance with the present invention. Referring to FIG. 5, in step 510 a user plays CD 460 on a conventional CD playback device for generating sound output and is greeted with a message. The contents of the message can be user specific. For example, a message to welcome the user and introduce the hearing training session can be conveyed as a greeting. Method 500 proceeds to step 515.

In step 515, a user plays the next track on CD 460. The track contains a training word unit including a first word 345 of a sentence 350. The first word 345 of the sentence 350 is played. For example, Word 3 marked under frequency-1 335 can be played initially. The Word 345 is played normally, as shown in individual hearing 230, so that the user hears it without the hearing aid and how the user expects to hear it. In the beginning, the word may sound like: “elephant.” Even though the person speaking the word “elephant” provides the correct frequency and amplitude, so that persons with normal hearing understand it as the word “elephant”, the user's poor hearing transmits to his brain a degraded frequency and amplitude. Over time, the user's brain learns this new frequency and amplitude as the word “elephant”, but a person of normal hearing would not recognize the word as “elephant”. Method 500 then proceeds to step 520.

In step 525, user plays the next track on CD 460. The first word 345 of the first sentence 350 is played again, now incorporating amplification factor 250. Thus, the word is played as the user would hear it with the hearing aid. In the beginning, word 345 may sound like: “elephenTT” with an exaggerated frequency “t” component, because that is how the word would sound through the hearing aid. Although the user might not understand the word initially, he can be trained to understand it by playing it repeatedly. Method 500 proceeds to step 525.

In step 525, user determines if he is satisfied with the way his brain hears and interprets the modified version of the word and sentence combination as played in step 520. If the user understands the word, he has learned it. If the user does not understand the word, he or she can replay the track until he is accustomed to the modified version of the word. If the user feels that he has learned the word, method 500 proceeds to step 530. If not, method 500 returns to step 515.

It is easily understood that individuals vary in speed of learning words, i.e., some learn words faster or slower than others. Therefore, in an alternative preferred embodiment of the invention, the test unit 115 analyzes the user's speed and ability to grasp words and provides this information for use in customizing and fitting a programmable hearing aid. This analysis can be performed iteratively, as part of an aural rehabilitation plan, such that an on-going analysis of the user's improvements in grasping words can be used to further fine-tune the user's hearing aid over time.

In a preferred embodiment, steps 520 and 525 are performed iteratively where the first word 345 is played adjusted, incorporating a fraction of the amplification factor 250 and then, after the user understands the word played with the fraction of the amplification factor, the word is then played adjusted, incorporating an incremented fraction of the amplification factor. The fraction of the amplification factor continues to be incremented in accordance with a predetermined incremental function, and based on the user becoming accustomed to the word with increased amplification, until the word is played adjusted incorporating the entire amplification factor. For example, if the amplification factor is 40 dB, the word is played adjusted first with amplification of 20 dB, then 30 dB and finally 40 dB.

Step 530 is performed following a determination by the user in step 525 that the word being played has been learned. In step 530, the user determines if he would like to review additional groups of words and sentences. If yes, method 500 returns to step 515; if not, method 500 ends.

In another preferred embodiment, the media 460 is a CD-ROM or equivalent and the system 460 records on such media training word units containing respective identified troublesome words with corresponding amplification factors and also test words for each of the training units. The identified troublesome words and the associated test words, together, are used to train an individual to hear and correctly recognize troublesome words prior to using a hearing aid. Referring to FIG. 4, content database 410 includes several test words for each of the troublesome words 345. The test words include words that are similar to a troublesome word and also include the troublesome word itself. For example, the troublesome word can be “bent” and the test words can include “bent”, “bean”, “been” and “bend”. The program 440, for each of the affected words 445 stored as a training word unit on the media .460, retrieves the corresponding test words from the database 410 and, using the drive 455, stores the test words on the media 460 as a test word list. The test word lists are linked to respective training word units on the media 460 that contain the corresponding troublesome word 345 with an amplification factor. The program 440 further includes, and causes the drive 455 to record onto the media 460, a playback application that a conventional computer containing a conventional monitor, mouse, keyboard and speakers can execute to generate sound output and display text using data stored on the media 460.

When the application on the media 460 is executed by the computer, for each training word unit on the media 460 including a troublesome word with a corresponding amplification factor, sound output of the troublesome word with the amplification factor is generated and the words of the linked test word list are displayed on the monitor. The individual performing hearing training using the media 460, who does not yet have a hearing aid, is then prompted on the monitor to select the word from the list of displayed test words that the individual believes was generated as sound output. If the individual selects the correct word, such as indicated by a keyboard or mouse entry, the application generates, using a different training word unit, sound output of a different troublesome word with the corresponding amplification factor and performs the same actions as above. If the user selects the incorrect word, the application highlights the correct word on the display and again generates sound output of the troublesome word with the amplification factor. Consequently, the troublesome words are re-enforced as part of hearing training so that the individual is trained to hear and recognize the troublesome words with the corresponding amplifications. This hearing training prepares the individual for hearing troublesome words using a hearing aid that the individual has ordered but yet to receive.

Thus, the present invention provides for creation of a customized hearing training product on a data storage media, which can be portable or otherwise easily accessible over communications networks, and where the media includes user-specific training words, generated from the user's hearing profile, and stored on the media to provide that normal or modified versions of the words are played back as part of hearing training and also can be selectively accessed by the user.

Although preferred embodiments of the present invention have been described and illustrated, it will be apparent to those skilled in the art that various modifications may be made without departing from the principles of the invention.

What is claimed is:

1. A method of customized hearing training comprising:
mapping a hearing loss profile for an individual based on hearing loss testing information for the individual;
identifying troublesome content from a troublesome word database based on the hearing loss profile, wherein the troublesome content includes at least one of a troublesome word and a sentence including the troublesome word; and storing a training word unit on a data storage media, wherein the training unit includes the troublesome content associated with a corresponding amplification factor for the individual and can be accessed to generate a sound output constituting a normal version of the content and a modified version of the content amplified by the corresponding amplification factor.

2. The method of claim 1, wherein the media includes a plurality of the training word units and wherein the amplification factor for a first of the training units includes a plurality of fractional amplification factors constituting different fractions of the corresponding amplification factor, wherein the first training unit can be accessed to generate sound outputs of the content of the first training unit having incremental levels of amplification.

3. A system for customized hearing training comprising:
a customized training means for testing an individual having hearing loss and mapping a hearing loss profile for the individual based on results of hearing loss testing;
wherein the training means identifies troublesome content from a troublesome word database based on the hearing loss profile, wherein the troublesome content includes at least one of a troublesome word and a sentence including the troublesome word; and
wherein the training means stores a training word unit on a data storage media, wherein the training unit includes the troublesome content associated with a corresponding amplification factor for the individual and can be accessed to generate a sound output constituting a normal version of the content and a modified version of the content amplified by the corresponding amplification factor.

4. The system of claim 3, wherein the data storage media includes at least one of a portable data storage media and a data storage media accessible over communications networks.

5. The system of claim 3, wherein the training means includes the troublesome word database.

6. The system of claim 3, wherein the media includes a plurality of the training word units and wherein the amplification factor for a first of the training units includes a plurality of fractional amplification factors constituting different fractions of the corresponding amplification factor, wherein the first training unit can be accessed to generate sound outputs of the content of the first training unit having incremental levels of amplification.

7. A method of customized hearing training comprising:
collecting frequency and amplitude hearing loss data for the individual by performing a frequency versus amplitude hearing test on the individual;
generating a hearing loss profile map including frequencies requiring amplification and associated amplification factors based on the frequency and amplitude data;
providing a troublesome word database, wherein the database includes a plurality of words, wherein each of the words includes at least one frequency component and is indexed in the database in accordance with the at least one frequency component;
generating pairs of training word units, wherein one of a first pair of the training units includes a troublesome word from the word database having at least one frequency component substantially equal to a frequency requiring amplification in the profile map, and wherein the other of the first pair of the training units includes the troublesome word and an amplification factor for the frequency requiring amplification; and
storing the training units on data storage units of at least one of a fixed and portable data storage media, wherein the respective pairs of the training units are stored on the media such that a normal version and a modified version of the word included in a pair of the training units can be generated as a sound output, wherein the normal version sound output is without any amplification and the modified version sound output includes selected amplification of the word based on the amplification factor.

8. The method of claim 7, wherein the other of the first pair of training units includes a plurality of fractional amplification factors constituting different fractions of the corresponding amplification factor, wherein the fractional amplification factors are accessible to generate sound outputs of the content of the first pair of training units having incremental levels of amplification.

9. The method of claim 7, wherein the fixed media is accessible over a communications networks.

10. The method of claim 7, wherein the portable media is a CD.

11. The method of claim 7, wherein the pairs of the training units are stored on the media such that the individual can selectively generate, for each of the pairs of the training units, the normal or modified version sound output of the word.

12. A data storage media for customized hearing training of an individual to use a hearing aid, the media including a plurality of training word units including troublesome content, wherein the content in the respective training units represents an association between troublesome content indexed by frequency in a troublesome word database and a hearing loss profile mapped from results of hearing loss testing on an individual, wherein the troublesome content includes at least one of a troublesome word and a sentence including the troublesome word, where the media further includes amplification factors respectively corresponding to the troublesome content of the training units and wherein the training units can be accessed to generate a sound output constituting a normal version of the content and a modified version of the content amplified by the corresponding amplification factor.

13. The data storage media of claim 12, wherein at least a first of the amplification factors includes a plurality of fractional amplification factors constituting different fractions of the first amplification factor, wherein the fractional amplification factors are accessible to generate sound outputs of the content corresponding to the first amplification factor at incremental levels of amplification.

14. A data storage media for customized hearing training of an individual to use a hearing aid, the media including a plurality of training word units including troublesome content, wherein the content in the respective training units represents an association between troublesome content indexed by frequency in a troublesome word database and a hearing loss profile mapped from results of hearing loss testing on an individual, where the media further includes amplification factors respectively corresponding to the troublesome content of the training units, wherein the media includes listings of test words linked to the respective corresponding training units, wherein the test words include the troublesome word of the corresponding training unit and similar sounding words, and wherein the training units can be accessed to generate a sound output constituting a normal version of the content and a modified version of the content amplified by the corresponding amplification factor and wherein the listing of the test words respectively corresponding to the training units can be accessed to generate a text display on a monitor.

15. The data storage media of claim 14 further comprising an executable application program, wherein the program, upon execution by a computer playback device, for at least a first of the training units, provides for generation of a sound output constituting a modified version of the content amplified by the corresponding amplification factor, provides for display of the test words corresponding to the first training unit on the monitor, provides for display on the monitor, with the test words, of a prompt for selection of the test word generated as the sound output, and provides for repeated generation of a sound output constituting a modified version of the content amplified by the corresponding amplification factor if the test word that is selected is not the troublesome word.
